(19) United States
(12) Patent Application Publication
(10) Pub. No.: US 2009/0131857 A1
(43) Pub. Date: May 21, 2009

(54) Method and apparatus for introducing a medicinal dose directly into a mammalian patient's cerebrospinal fluid

(76) Inventor: Mark Geiger, Laguna Niguel, CA (US)

Correspondence Address: RAYMOND R. MOSER JR., ESQ.;MOSER IP LAW GROUP, 1030 BROAD STREET, 2ND FLOOR, SHREWSBURY, NJ, 07702, US

(21) Appl. No.: 11/986,301

(22) Filed: Nov. 20, 2007

(51) Int. Cl. A61M 5/31; A61K 38/43; A61K 31/145

(52) U.S. Cl. 604/28; 424/94.1; 514/665; 604/246

(57) ABSTRACT

A method and apparatus for introducing a medicinal dose directly into a mammalian patient's cerebrospinal fluid. One embodiment of the present invention comprises implanting a first and second device in a mammalian patient to administer the medicinal dose. The first device comprises a ventricular catheter, a reservoir with a built-in one-way valve, and a drug port with pump, wherein the three components are in fluid communication. The second device comprises a drug port-catheter system in fluid contact with lumbar sub-arachnoid space.

### BACKGROUND OF THE INVENTION

1. Field of the Invention

Embodiments of the present invention generally relate to therapeutic mechanisms for treating brain disorders, and more particularly, to a method and apparatus for introducing a medical dose directly into a mammalian patient's cerebrospinal fluid.

2. Description of the Related Art

Most drugs that are designed to treat brain diseases, such as Alzheimer's Disease (AD), encounter the difficulty of getting across the Blood-Brain-Barrier (BBB). The Blood-Brain-Barrier is the body's natural central nervous system defense mechanism. The Blood-Brain-Barrier is very effective in restricting the movement of certain molecules to the brain. Therefore, infections of the brain are quite rare. However, this same effective protection makes the treatment of brain infections or diseases that do occur very difficult. That is, the Blood-Brain-Barrier prevents therapeutic drugs that may be introduced into the blood stream from reaching the brain in the same manner in which harmful substances or infections are prevented from reaching the brain. Hence, drugs targeting brain diseases are typically administered to a patient in higher doses than what is actually needed to remedy the diseases. Thus, only a fraction of a systemic or intravenous (IV) dose of a drug targeting a brain disease would be required if the drug could be introduced directly into the cerebrospinal fluid (CSF) that is in and around a patient's brain and spine.

Accordingly, there exists the need for a method to overcome or circumvent the body's natural central nervous system defense mechanism to efficiently administer drugs or other therapeutic substances to the brain for the treatment of various brain diseases or infections.

### SUMMARY OF THE INVENTION

The present invention generally relates to a method and apparatus for introducing a medicinal dose directly into a mammalian patient's cerebrospinal fluid. One embodiment of the present invention comprises implanting a first device and second device in a mammalian patient. The first device comprises a ventricular catheter, where the catheter is in fluid contact with the lateral ventricle of the patient; a reservoir with a built-in one-way valve, wherein the reservoir is implanted subcutaneously under the scalp; and a drug port with pump, wherein the three components are in fluid communication. The second device comprises a drug port-catheter system in fluid contact with lumbar sub-arachnoid space; filling the drug port with a medicinal dose; waiting for a therapeutically sufficient period of time for the medicinal dose to take affect; removing cerebrospinal fluid from the lumbar sub-arachnoid space through the port-catheter system; infusing a fluid that mimics cerebrospinal fluid in an amount about equal to the amount removed through the subcutaneous reservoir to avoid a significant reduction in intracranial pressure; and accessing and refilling the drug port with the medicinal dose when another medicinal dose is required.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 is a drawing of a subcutaneous reservoir and ventricular catheter system that depicts certain aspects of various embodiments of the present invention.

FIG. 2 is a drawing of a subcutaneous reservoir and lumbar catheter system that depicts certain aspects of various embodiments of the present invention.

FIG. 3 is a drawing of a subcutaneous reservoir, drug pump system, and lumbar catheter system that depicts certain aspects of various embodiments of the present invention.

While the invention is described herein by way of example using several embodiments and illustrative drawings, those skilled in the art will recognize that the invention is not limited to the embodiments of drawing or drawings described. It should be understood that the drawings and detailed description thereto are not intended to limit the invention to the particular form disclosed, but on the contrary, the invention is to cover all modification, equivalents and alternatives falling within the spirit and scope of the present invention as defined by the appended claims. The headings used herein are for organizational purposes only and are not meant to be used to limit the scope of the description or the claims. As used throughout this application, the word “may” is used in a permissive sense (i.e., meaning having the potential to), rather than the mandatory sense (i.e., meaning must). Similarly, the words “include,” “including,” and “includes” mean including, but not limited to. Further, the word “a” means at least one.

### DETAILED DESCRIPTION

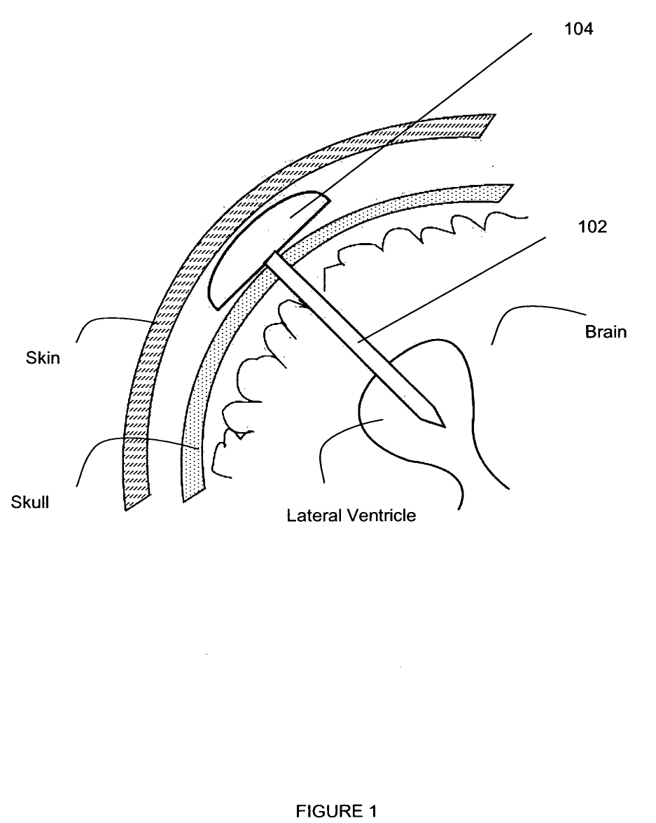

FIG. 1 is a drawing of a subcutaneous reservoir and ventricular catheter system that depicts certain aspects of various embodiments of the present invention.

As shown in FIG. 1, reservoir 104 is depicted implanted under a patient's scalp. Thus, the reservoir is labeled subcutaneous reservoir 104. One end of ventricular catheter 102 is in fluid contact the subcutaneous reservoir. The other end of the ventricular catheter is in fluid contact with the lateral ventricle of the patient's brain. Hence the lateral ventricle of the patient's brain is in fluid communication with the subcutaneous reservoir.

According to one embodiment of the present invention, subcutaneous reservoir 104 may be injected with a medicinal dose of an AD drug (e.g., NeuroChems's Alzhemed™, as reported in “7 ways to Save a Brain”, Newsweek Special Issue, 2005) that attracts harmful protein (e.g., A-Beta and Tau). The drug may be injected through the patient's scalp into the subcutaneous reservoir using, for example, a needle and syringe. The drug, then, may be infused through the ventricular catheter into the lateral ventricle of the brain and thus directly into the cerebrospinal fluid. Because the medicinal dose is administered in the manner described, circumventing the blood-brain-barrier, the dose amount may be reduced to a level appropriate for treating a targeted disease or brain disorder. There would be no need for a larger dose amount as would be required when the drug has to overcome the blood-brain-barrier to effectuate treatment.

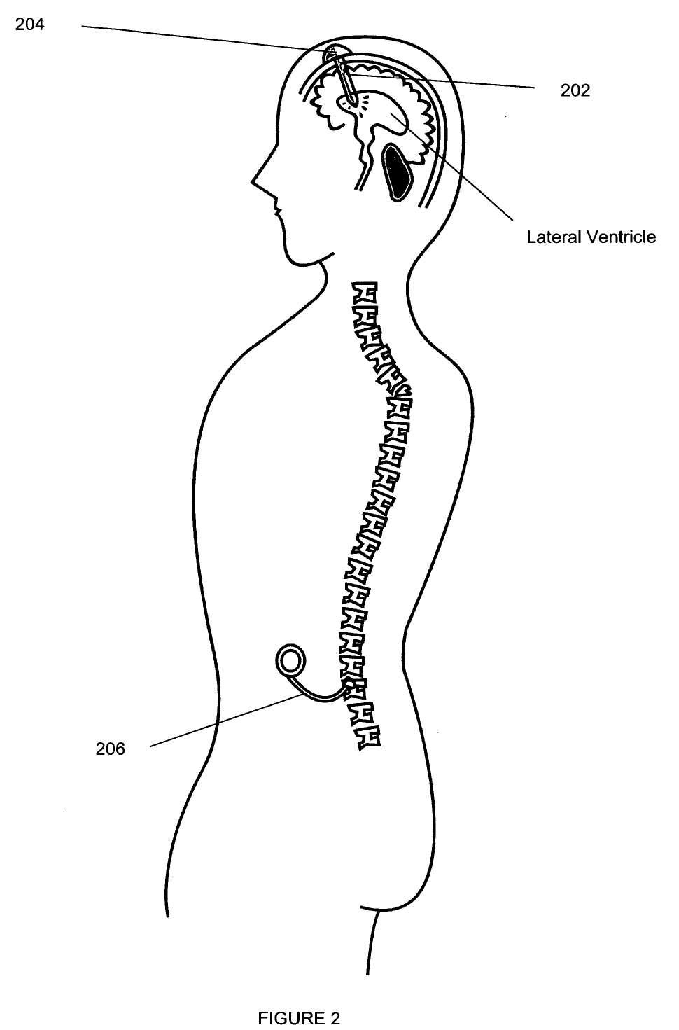

FIG. 2 is a drawing of a subcutaneous reservoir and lumbar catheter system that depicts certain aspects of various embodiments of the present invention.

As shown in FIG. 2, reservoir 204 is implanted under a patient's scalp. The reservoir is depicted equipped with two chambers. At one end, dual lumen catheter 202 is in fluid contact with the dual chamber reservoir. A first lumen of the dual lumen catheter is in fluid contact with a first chamber of the dual chamber reservoir. A second lumen of the dual lumen catheter is in fluid contact with the second chamber of the dual chamber reservoir. The other end of the dual lumen catheter is in fluid contact with the lateral ventricle of the patient's brain. Thus the lateral ventricle is in fluid communication with the dual chamber reservoir. Alternately (not shown), two single lumen catheters may be utilized instead of the dual lumen catheter. The dual or single lumen catheter may be comprised of a silicone elastomer.

According another embodiment of the present invention, the first chamber of subcutaneous reservoir 204 may be injected with a medicinal dose of an AD drug (e.g., NeuroChems's Alzhemed™, reported in “7 ways to Save a Brain”, Newsweek Special Issue, 2005) that attracts harmful protein (e.g., A-Beta and Tau) or enzyme that eats/digests A-Beta and/or other harmful proteins. The drug may be injected through the patient's scalp into the first chamber of the subcutaneous reservoir using, for example, a needle and syringe. The drug, then, may be infused through the first lumen of the dual lumen catheter into the lateral ventricle of the brain and thus directly into the cerebrospinal fluid.

After a therapeutically sufficient period of time for the medicinal dose to take effect has elapsed, cerebrospinal fluid may be removed through the second chamber of the dual chamber reservoir via the second lumen of the dual lumen catheter. Drugs such as Alzhemed™ are designed to attract harmful proteins (e.g., A-Beta and Tau). By using such drugs in the manner just described, the deposit of harmful proteins into a patient's brain tissue may be avoided. Also again, administering drugs across or behind the blood-brain-brain barrier as just described reduces the amount of a medicinal dose required to therapeutically treat a targeted brain disorder.

Alternate to removing cerebrospinal fluid through a second chamber of a dual chamber reservoir (e.g., dual chamber reservoir 204) some embodiments of the present invention comprises, instead, removing cerebrospinal fluid from a patient's lumbar subarachnoid space through an implanted lumbar catheter and reservoir system which is in fluid contact with the patient's lumbar subarachnoid space (e.g., lumbar catheter and reservoir system 206).

In accordance with various embodiments of the present invention, the removed cerebrospinal fluid may be replaced via the first chamber of dual chamber reservoir (e.g., dual chamber reservoir 204) with normal saline or a fluid that mimics cerebrospinal fluid. By replacing the removed cerebrospinal fluid, a significant drop in the patient intra-cranial pressure can be avoided.

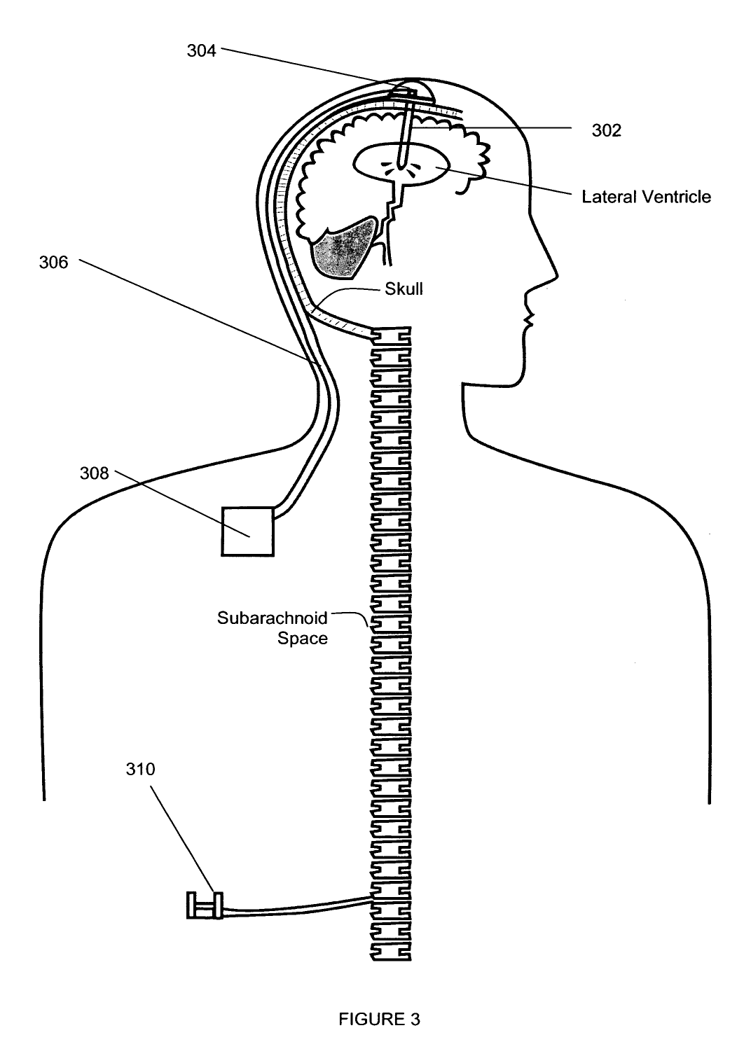

FIG. 3 is a drawing of a subcutaneous reservoir, drug pump system, and lumbar catheter system that depicts certain aspects of various embodiments of the present invention.

As shown in FIG. 3, reservoir 304 is implanted under a patient's scalp. The reservoir is depicted equipped with a one-way valve. At one end, catheter 306 is in fluid contact with reservoir 304. At the other end catheter 306 is in fluid contact with drug pump 308. Additionally, one end of ventricular catheter 302 is also in fluid contact with reservoir 304. The other end of ventricular catheter 302 is in fluid contact with the lateral ventricle of the patient's brain. Consequently, the drug pump system is in fluid communication with the patient's lateral ventricle.

According to yet another embodiment of the present invention, a medicinal dose of an AD drug may be injected into drug pump system 308. The drug may then be infused by the pump system through reservoir 304 to the lateral ventricle via ventricle catheter 302 and thus directly into the cerebrospinal fluid.

As with various other embodiments of the present invention, cerebrospinal fluid may be removed from the patient's lumbar subarachnoid space through an implanted lumbar catheter and reservoir system which is in fluid contact with the patient's lumbar subarachnoid space (e.g., port with lumbar catheter system 310). Again, such removal of cerebrospinal fluid is undertaken after a therapeutically sufficient period of time for the medicinal dose to take effect has elapsed.

While the foregoing is directed to embodiments of the present invention, other and further embodiments of the invention may be devised without departing from the basic scope thereof, and the scope thereof is determined by the claims that follow.

1. A method for introducing a medicinal dose directly into a mammalian's cerebrospinal fluid comprising:
a) implanting a first device comprising
i) a ventricular catheter, where the catheter is in fluid contact with the lateral ventricle of the patient;
ii) a reservoir with a built-in one-way valve, wherein the reservoir is implanted subcutaneously under the scalp; and
iii) a drug port with pump into the patient, wherein the three components are in fluid communication;
b) implanting a second device comprising a port-catheter system in fluid contact with lumbar sub-arachnoid space;
c) filling the drug port with the medicinal dose;
d) waiting for a therapeutically sufficient period of time for the medicinal dose to take affect;
e) removing cerebrospinal fluid from the lumbar sub-arachnoid space through the port-catheter system;
f) infusing a fluid that mimics cerebrospinal fluid in an amount about equal to the amount removed;
g) injecting cerebrospinal fluid into the subcutaneous reservoir to avoid a significant reduction in intracranial pressure; and
accessing and refilling the drug port with the medicinal dose when another medicinal dose is required.

2. The method of claim 1 wherein the medicinal dose is an enzyme that eats or digest A-Beta, or other harmful proteins.

3. The method of claim 1 wherein the medicinal dose is a drug that attracts harmful proteins.

4. The method of claim 3 wherein the drug is Alzhemed™.

5. A method for introducing a medicinal dose directly into a mammalian patient's cerebrospinal fluid comprising:
a) implanting a first device comprising
i) ventricular catheter with at least two lumens wherein the catheter is in fluid contact with the patient's lateral ventricle;
ii) a vessel with at least two chambers wherein the vessel is in fluid contact with the ventricular catheter and is subcutaneously implanted;
b) filling the one chamber the medicinal dose wherein the dose travels via a first lumen of the catheter to the lateral ventricular;
c) waiting for a therapeutic sufficient period of time for the medicinal dose to take affect;
d) removing cerebrospinal fluid through a second lumen of the catheter;
e) infusing a fluid that mimics cerebrospinal fluid in an amount about equal to the amount of the removed cerebrospinal fluid into one of the subcutaneous chambers to avoid a significant reduction in intracranial pressure; and
f) accessing and refilling the dome with the medicinal dose when another medicinal dose is required.

6. The method of claim 5 wherein the medicinal dose is an enzyme that eats harmful proteins.

7. The method of claim 5 wherein the medicinal dose is a drug that attracts harmful proteins like A-Beta and Tau.

8. The method of claim 7 wherein the drug is Alzhemed™.

9. The method of claim 5 wherein the dome is comprised of silicone.

10. The method of claim 5 wherein the ventricular catheter is comprised of a silicone elastomer.

11. A method for introducing a medicinal dose into a mammalian patient's cerebrospinal fluid comprising:
a) implanting a device comprising:
i) ventricular catheter in fluid contact with the lateral ventricle; and
ii) a chamber in fluid contact with the ventricular catheter wherein the chamber is subcutaneously implanted in the scalp of the patient's head;
b) filling the chamber with a medicinal dose;
c) waiting for a therapeutic sufficient period of time for the medicinal dose to take effect;
d) removing cerebrospinal fluid through the chamber;
e) infusing a fluid that mimics cerebrospinal fluid in an amount about equal to the amount removed from the chamber to avoid a significant reduction in intracranial pressure; and
accessing and refilling the dome with the medicinal dose when another medicinal dose is required.

12. The method of claim 11 wherein the medicinal dose is an enzyme that eats harmful proteins.

13. The method of claim 11 wherein the medicinal dose is a drug that attracts harmful proteins like A-Beta and Tau.

14. The method of claim 13 wherein the drug is Alzhemed.

15. A device for introducing a medicinal dose directly into a patient's cerebrospinal fluid comprising:
a) a ventricular catheter;
b) a chamber with a built-in one-way valve wherein the chamber is in fluid contact with the catheter; and
c) a drug port with pump wherein the pump is in fluid contact with the chamber.

16. A device for introducing a medicinal dose directly into a mammalian patient's cerebrospinal fluid comprising:
a) a ventricular catheter; and
b) a reservoir with at least two chambers wherein the chambers are in fluid contact with the catheter and wherein at least one of the chambers contains a therapeutic useful in the treatment of Alzheimer's Disease.

17. The device of claim 16 wherein the ventricular catheter further comprises a first lumen and a second lumen.
